(19) United States
(12) Patent Application Publication
(10) Pub. No.: US 2005/0171088 A1
(43) Pub. Date: Aug. 4, 2005

(54) Treatment of psychoses with dibenzothiazepine antipsychotic

(75) Inventors: Brian Ault, Wilmington, DE (US); Gilbert Block, Wilmington, DE (US); Martin Brecher, Wilmington, DE (US); Wayne MacFadden, Wilmington, DE (US); Robin McCoy, Wilimington, DE (US); Margaret Minkwitz, Wilmington, DE (US); Jamie Mullen, Wilmington, DE (US); Ellis Wilson, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB

(21) Appl. No.: 11/043,622

(22) Filed: Jan. 26, 2005

Related U.S. Application Data
(60) Provisional application No. 60/540,618, filed on Jan. 30, 2004.

(51) Int. Cl. A61K 31/554

(52) U.S. Cl. 514/211.13

(57) ABSTRACT

The present invention provides methods for treating depression symptoms associated with bipolar disorder.

### FIELD OF THE INVENTION

The present invention relates to methods using a dibenzothiazepine antipsychotic.

### BACKGROUND

The bipolar disorders are mood disorders in which a disturbance in mood is the predominant feature. Bipolar I disorder is characterized by one or more manic or mixed episodes, usually accompanied by major depressive episodes. Bipolar II disorder is characterized by one or more major depressive episodes accompanied by at least one hypomanic episode. Bipolar depression refers to the major depressive episodes that occur with bipolar I and II disorder.

The prevalence of bipolar disorder is estimated to be 1 to 3.5%, evenly divided between men and women. The length of time between onset and symptoms and proper diagnosis and treatment is approximately 10 years. It is estimated that only 60% of those suffering from a bipolar disorder are receiving appropriate pharmacotherapy.

Although there is extensive and emerging literature guiding the treatment of the manic phase of bipolar I disorder as well as many approved compounds for the treatment of unipolar depression, the treatment of bipolar depression has not been widely studied and treatment guidelines are in their infancy. The use of currently available antidepressants for monotherapy for bipolar depression is often problematic as they may increase the “switch” into hypomania or mania from depression, or increase cycle acceleration. Further, patients can experience treatment-emergent mania with antidepressant monotherapy. The adjunctive use of mood stabilizing medications such as lithium carbonate (LiCO₃) is common and may decrease the likelihood of these complications.

Evidence indicates that medications with mood stabilizing properties which produced low levels of mania, hypomania, or cycle acceleration may be useful as monotherapy in the treatment of bipolar depression. The antiepileptic lamotrigine produced improvement in HAM-D and MADRS scores in a 7-week, double-blind, placebo controlled trial for the patients who completed this study (Calabrese 1999). More recently, the anti-manic agent divalproex demonstrated numerical improvement over placebo in the percentage of patients with bipolar depression having a 50% reduction in the HAM-D scores without mania in an 8 week trial (Sachs, 2001) but this difference was not statistically significant. Lithium carbonate, also approved for the treatment of mania, has been demonstrated to be effective as a monotherapeutic agent in approximately 50% of patients with bipolar depression (Bauer). However, there are limitations to the use of the above therapies.

### DETAILED DESCRIPTION

Quetiapine fumarate is described in U.S. Pat. No. 4,879,288, which is incorporated herein by reference. Quetiapine fumarate (quetiapine) is a dibenzothiazepine derivative and is designated chemically as 2-[2-(4-dibenzo [b,ƒ] [1,4]thiazepin-11-yl-1-piperazinyl)ethoxy]-ethanol fumarate.

However, applicants have reached surprising results that indicate the success of quetiapine in treating depression states. Recent clinical studies have revealed previously unrecognized pharmacological properties which suggest that quetiapine is useful in treating depression associated with bipolar disorder. Further, quetiapine was found to be well-tolerated in the treatment of bipolar depression with a low incidence of EPS (extrapyramidal symptoms), prolactin, sexual dysfunction and weight gain. Additionally, quetiapine was not associated with treatment-emergent mania in the treatment of bipolar depression and treatment resulted in a low rate of treatment-emergent mania.

It has now been discovered that quetiapine or a pharmaceutically acceptable salt thereof is an effective treatment of the depression symptoms associated with one or more mood disorders.

Certain embodiments of the invention include a method for treating depression symptoms associated with one or more mood disorders comprising administering to a patient a therapeutically effective amount of a pharmaceutical composition comprising a pharmaceutically acceptable carrier and a compound of Formula (I):

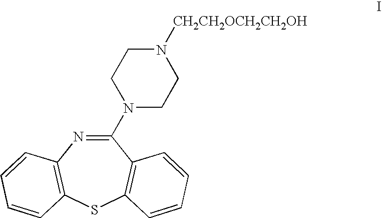

Certain embodiments of the method include the use of a compound of quetiapine or a pharmaceutically acceptable salt thereof, for the preparation of a medicament for treating depression symptoms associated with one or more mood disorders in a patient.

Other embodiments of the method include the use of a compound of quetiapine or a pharmaceutically acceptable salt thereof, for the preparation of a medicament for treating depression symptoms associated with bipolar disorder in a patient.

The present invention relates to a method for treating one or more mood disorders by administering quetiapine. The structure of quetiapine is shown in Formula I:

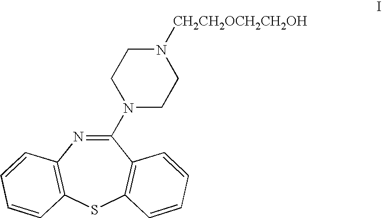

One embodiment of the invention provides a method which comprises administering quetiapine or a pharmaceutically acceptable salt to a patient for the treatment of depression symptoms associated with one or more mood disorders.

Another embodiment of the invention provides a method which comprises administering quetiapine fumarate to a patient for the treatment of depression symptoms associated with bipolar disorder. Another embodiment of the invention provides a method which comprises administering quetiapine fumarate to a patient for the treatment of depression symptoms associated with bipolar I disorder.

Another embodiment of the invention provides a method which comprises administering quetiapine fumarate to a patient for the treatment of depression symptoms associated with bipolar II disorder.

Another embodiment of the invention provides a method which comprises administering quetiapine fumarate to a patient for the treatment of depression symptoms associated with bipolar depression.

The term “therapeutically effective amount” as used herein means an amount of the compound which is effective in treating the named disorder or condition.

In one embodiment, bipolar depression may be treated by administering quetiapine to a patient in a dosage ranging from about 300 mg/day to about 600 mg/day.

Applicants have discovered that quetiapine is more effective than placebo and well tolerated for the treatment of depressive episodes in patients with one or more mood disorders. Applicants have further discovered that quetiapine is more effective than placebo and well tolerated for the treatment of depressive episodes in patients with bipolar depression. Moreover, quetiapine is more effective than placebo and well tolerated for the treatment of anxiety symptoms, reduced sleep quality and reduced quality of life in patients with bipolar disorder.

The following examples provided are not meant to limit the invention in any manner and are intended for illustrative purposes only.

### EXAMPLES

The results of a monotherapy study demonstrates the therapeutic value of the use of quetiapine fumarate in the treatment of patients with bipolar depression.

### Study

The study was a multicenter, 8 week, double-blind, randomized, placebo-controlled, double-dummy trial of the use of quetiapine fumarate in the treatment of patients with bipolar depression conducted in 539 subjects with 511 patients in ITT population. The treatment was with quetiapine or placebo. There were 43% male and 57% female patients. The demographics also included 67% bipolar I and 33% bipolar II.

Some of the key inclusion criteria: Meets DSM-IV criteria for bipolar disorder I or bipolar II, most recent episode depressed (296.5× and 296.89×), confirmed by a modified Structured Clinical Interview for DSM-IV (SCID); (2) current episode of depression >4 weeks; Some of the key exclusion criteria: at screen and baseline: HAMD-D (17-item) total score ≧20; HAM-D item 1 (depressed mood) score ≧2; previous treatment with an adequate course of more than 2 antidepressants for their current episode OR treatment for greater than 12 months; >12 on the YMRS (i.e., no mixed episodes); current (or within past 6 months) Axis I disorder other than bipolar disorder.

### Dosing

Quetiapine was titrated in a blinded manner to a total daily dose of about 300 mg/day by Day 4 in the 300-mg/day treatment group and to a total daily dose of about 600 mg/day by Day 8 in the 600-mg/day treatment group. Thereafter, oral doses of quetiapine fumarate were administered in a blinded fashion once daily in a total daily dose of about 300 or about 600 mg/day.

TITRATION SCHEDULE

| | Wash- | | | | | | | | | | |
|---|---|---|---|---|---|---|---|---|---|---|---|
| | out | | | | | | | | | | |
| Rx | 7-28 | DAY | | | | | | | | | |
| Group | days | 1 | 2 | 3 | 4 | 5 | 6 | 7 | 8 | 9-56 | 57 |
| 300 mg | | 50 | 100 | 200 | 300 | 300 | 300 | 300 | 300 | 300 | |
| HS dosing | | | | | | | | | | | |
| 600 mg | | 50 | 100 | 200 | 300 | 400 | 400 | 400 | 600 | 600 | |
| HS dosing | | | | | | | | | | | |

| | |
|---|---|
| PBO | ---------------------------------------------------------→ |

Primary endpoints were determined by MADRS (Montgomery/Asberg Depression Rating Scale (MADRS) with change from baseline to final assessment. Secondary endpoints evaluated by HAM-D (Hamilton Rating Scale for Anxiety), CGI-S (Clinical Global Impression-Severity), CGI-C (Clinical Global Impression-Change) change from baseline: incidence of treatment-emergent mania compared to placebo, effect of quetiapine on anxiety and the safety and tolerability of quetiapine in the treatment of patients with bipolar depression. Exploratory endpoints included efficacy of quetiapine on sleep quality (as determined through the Pittsburgh Sleep Quality Index (PSQI)), efficacy of quetiapine on the overall quality of life (through the Quality of Life Enjoyment and Satisfaction Questionnaire (Q-Les-Q, short form).

Results

Change in MADRS, Bipolar I's & II's (ITT (Intent to Treat) Population)

| | QTP 600 mg | QTP 300 mg | Placebo |
|---|---|---|---|
| | Mean (SD) | Mean (SD) | Mean (SD) |
| | N | N | N |
| Bipolar I | −18.2 (11.0) | −17.1 (9.7) | −9.5 (10.6) |
| | 114 | 116 | 112 |
| Bipolar II | −13.9 (10.2) | −15.2 (10.2) | −12.2 (11.0) |
| | 56 | 56 | 57 |

MADRS/HAM-D Results: ITT and Completer Populations (PLA=Placebo)

| | | MADRS | | | HAM-D | | |
|---|---|---|---|---|---|---|---|
| Pop | Result | 600 | 300 | PLA | 600 | 300 | PLA |
| ITT | N | 170 | 172 | 169 | 170 | 172 | 169 |
| | Baseline | 30.3 | 30.3 | 30.6 | 24.7 | 24.5 | 24.6 |
| | Change | −16.8 | −16.5 | −10.4 | −13.9 | −13.4 | −8.6 |
| OC | N | 95 | 117 | 94 | 97 | 119 | 99 |
| | Change | −20.3 | −18.6 | −13.2 | −17.0 | −15.0 | −10.6 |

Q-LES-Q—Week 4 & 8:

| | 600 mg | 300 mg | Placebo |
|---|---|---|---|
| | Mean (SD) | Mean (SD) | Mean (SD) |
| Baseline | 34.0 (8.1) | 36.0 (7.9) | 34.3 (7.3) |
| Week 4 (Δ) | 11.0 (10.7)* | 8.6 (9.6)^{a} | 6.0 (9.2) |
| Week 8 (Δ) | 16.3 (10.3)* | 11.7 (10.4)^{a} | 8.9 (10.1) |
| Week 8 (Δ) LOCF | 12.2 (11.6)* | 10.2 (10.7)* | 6.8 (10.0) |

*P < 0.001; ^{a}P < 0.05

PSQI—Week 4 & 8

| | 600 mg | 300 mg | Placebo |
|---|---|---|---|
| | Mean (SD) | Mean (SD) | Mean (SD) |
| Baseline | 11.8 (4.2) | 11.3 (3.8) | 11.7 (3.8) |
| Week 4 (Δ) | −5.3 (4.9)* | −4.7 (4.2)* | −2.5 (4.2) |
| Week 8 (Δ) | −6.4 (4.3)* | −5.3 (4.3)* | −3.8 (4.1) |
| Week 8 (Δ) LOCF | −5.5 (4.8)* | −5.1 (4.3)* | −3.0 (4.2) |

*P < 0.001; LOCF: Last Observation Carrier Forward

Efficacy Summary

Efficacy against depressive symptoms in both doses from Day 8 on (p<0.001) (MADRS and HAM-D). 20% advantage over placebo for MADRS Responder analysis; MADRS effect size 0.6 (Bipolar I & II); 20% advantage over placebo in remission analysis; MADRS effect size: 0.6 (Bipolar I & II). Efficacy in anxiety symptoms (HAM-A) in both doses from Day 8 on (p<0.01). Clinical Improvement (CGI) in both doses from Day 8 on (p<0.001). Significant results in patient reported outcomes (PSQI and Q-LES-Q).

Treatment-Emergent Mania

Criteria for Emergent Mania (any one of the following): AE (Adverse Event) or SAE (Serious Adverse Event) of Mania. Withdrawal for AE of mania. YMRS (Young Mania Rating Scale) ≧16 on 2 consecutive or final assessment. These results suggest that quetiapine is not associated with treatment-emergent mania (“switching”) in the treatment of bipolar depression.

| 600 mg | 300 mg | Placebo |
|---|---|---|
| 4 (2.4%) | 6 (3.5%) | 7 (4.1%) |

Quetiapine was found to also exhibit efficacy in a broad range of symptom domains in bipolar depression, including anxiety and reduction in sleep quality.

Sleep Week 8 LOCF

| | 600 mg | 300 mg | Placebo |
|---|---|---|---|
| Component | Mean (SD) | Mean (SD) | Mean (SD) |
| Sleep quality | 0.8 (0.8) | 1.0 (0.8) | 1.5 (0.9) |
| Sleep latency | 1.3 (1.1) | 1.3 (1.0) | 1.8 (1.1) |
| Sleep duration | 0.5 (0.8) | 0.7 (0.8) | 1.2 (1.0) |
| Sleep efficiency | 0.7 (1.1) | 0.6 (1.0) | 1.1 (1.1) |
| Sleep disturbance | 1.2 (0.7) | 1.1 (0.6) | 1.4 (0.7) |
| Sleep medication | 0.4 (0.9) | 0.4 (0.9) | 0.3 (0.8) |
| Sleep dysfunction | 1.4 (0.9) | 1.3 (0.9) | 1.5 (0.8) |

Anxiety

Mean baseline levels of anxiety measured by HAM-A score were similar across treatment groups: 18-6-18.9. Patients taking quetiapine about 300 and about 600 mg/day had significantly (P<0.05) greater improvement in mean HAM-A score vs. placebo at every assessment starting with the first evaluation (Day 8) and sustained through endpoint (Week 8) (−8.6 and −8.7 vs −5.5).

Safety Summary

No unexpected AE trends; low rate of emergent mania; comparable across all groups; no statistical difference in completion rates, dose related trends, increase in withdrawals for AE, reduction in withdrawals for lack of effect. Small dose related changes in weight. Accordingly, quetiapine was found to be safe and effective for the treatment of bipolar depression, effective in the treatment of anxiety symptoms associated with bipolar depression, effective in improving the quality of life and sleep quality in patients with bipolar depression.

1. A method for treating depression symptoms of one or more mood disorders in a patient comprising administering to a patient a therapeutically effective amount of quetiapine or a pharmaceutically acceptable salt thereof.

2. The method according to claim 1, wherein the pharmaceutically acceptable salt of quetiapine is quetiapine fumarate.

3. The method according to claim 1, wherein the depression symptom is anxiety.

4. The method according to claim 1, wherein the depression symptom is reduced sleep quality.

5. The method according to claim 1, wherein the depression symptom is reduced quality of life.

6. The method according to claim 1 wherein said quetiapine is administered at a dose from about 300 mg/day to about 600 mg/day for a patient.

7. The method according to claim 1 wherein said quetiapine is administered at a dose of about 300 mg/day.

8. The method according to claim 1 wherein said quetiapine is administered at a dose of about 600 mg/day.

9. The method according to claim 1 wherein said quetiapine is administered once daily.

10. The method according to claim 1, wherein the amount of quetiapine results in a low rate of treatment-emergent mania.

11. The method according to claim 1, wherein said mood disorder is bipolar disorder.

12. A method according to claim 11, wherein said bipolar disorder is bipolar I.

13. A method according to claim 11, wherein said bipolar disorder is bipolar II.

14. A method for treating depression symptoms of bipolar disorder comprising administering to a patient a therapeutically effective amount of a compound of Formula (I): 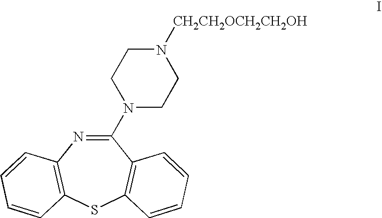
or a pharmaceutically acceptable salt thereof.

15. A method according to claim 14, wherein the pharmaceutically acceptable salt of quetiapine is quetiapine fumarate.

16. A monotherapeutic method of treating a patient for the depression symptoms of one or more mood disorders comprising administering to the patient a therapeutically effective amount of quetiapine or a pharmaceutically acceptable salt thereof.

17. A method according to claim 16, wherein the mood disorder is bipolar disorder.

18. A method according to claim 16, wherein the bipolar disorder is bipolar I.

19. A method according to claim 16, wherein the bipolar disorder is bipolar II.

20. A monotherapeutic method of treating a patient for the depression symptoms of bipolar disorder comprising administering to the patient a therapeutically effective amount of 2-[2-(4-dibenzo [b,ƒ] [1,4]thiazepin-11-yl-1-piperazinyl)ethoxy]-ethanol fumarate.

21. A method for the treatment of depression symptoms by administering to a patient an antidepressant amount of a compound selected from quetiapine and a pharmaceutically acceptable salt thereof.

22. The use of a compound of quetiapine or a pharmaceutically acceptable salt thereof, for the preparation of a medicament for treating depression symptoms associated with one or more mood disorders in a patient.

23. The use of a compound of quetiapine or a pharmaceutically acceptable salt thereof, for the preparation of a medicament for treating depression symptoms associated with bipolar disorder in a patient.
